(19) United States
(12) Patent Application Publication
(10) Pub. No.: US 2022/0331427 A1
(43) Pub. Date: Oct. 20, 2022

(54) NON-INVASIVE NEAR-INFRARED LIGHT-CONTROLLED NANOMATERIAL FOR TREATMENT OF DIABETES

(71) Applicant: SOOCHOW UNIVERSITY, Suzhou (CN)

(72) Inventors: Jian LIU, Suzhou (CN); Jun YAN, Suzhou (CN)

(21) Appl. No.: 17/616,649

(22) PCT Filed: Dec. 30, 2020
(86) PCT No.: PCT/CN2020/141134

§ 371 Date: Dec. 4, 2021

(30) Foreign Application Priority Data
Jul. 28, 2020 (CN) 202010738153.1

(51) Int. Cl. A61K 41/00; A61K 47/69; A61K 47/60; A61K 47/54; A61P 3/10

(52) U.S. Cl. CPC A61K 41/0038; A61K 47/6923; A61K 47/60; A61K 47/554; A61P 3/10; B82Y 5/00

(57) ABSTRACT

The present invention provides a non-invasive near-infrared light-controlled nanomaterial for the treatment of diabetes and use of an upconversion fluorescent nanomaterial in the preparation of a tool for the treatment of diabetes, wherein the upconversion fluorescent nanomaterial includes an inorganic nanomaterial doped with rare earth elements, and a layer of water-soluble polymer and molecules targeting liver cells, which is on the surface of the nanomaterial. In the treatment of diabetes, there is no need to surgically implant invasive optical fibers in animals, and the upconversion nanomaterial in an organism is excited by near-infrared light with high tissue penetrability. The upconversion material converts the light of near-infrared band into visible light, to activate light-sensitive proteins. This enables the remote control of intracellular glucose metabolism-related signaling pathways independent of insulin with high temporal-spatial resolution, to promote the glycogen synthesis, inhibit the gluconeogenesis, and lower the blood glucose level.

### FIELD OF THE INVENTION

The present invention relates to the field of medical research of diabetes, and more particularly to a non-invasive near-infrared light-controlled nanomaterial for the treatment of diabetes.

### DESCRIPTION OF THE RELATED ART

Optogenetics is a new technology that combines the genetics and optics. In the past decades, optogenetics has achieved great progress in many research fields, including neuroscience, tumor therapy, signaling pathway research, and exosome engineering. To enrich the toolbox of optogenetic research, scientists have developed light-sensitive protein elements including rhodopsins, xanthopsins, phytochromes, and cryptochromes. In the optogenetics, target cells are genetically engineered, to implant corresponding light-sensitive proteins, and then the light-sensitive proteins in the target cells in a target area are precisely activated by a light-controlled method, to regulate the cell functions, thereby changing the physiological state of an individual.

The currently used optogenetic proteins only respond to light in the visible band (such as violet, blue, green, and yellow light). Therefore, due to the poor tissue penetrability of short-wavelength photons, the use of optogenetics in a living organism is greatly restricted. In the traditional optogenetic research, an optical device such as optical fiber is often implanted in animals to transmit optical signals. This not only leads to the higher mortality of experimental animals, but also causes the surviving animals to suffer from the implanted optical device throughout the experiment. The behavior of the animals will also be restricted by the optical fiber. As a result, great uncertainty is brought to the experimental results (especially in animal behavior experiments). Researchers have tried to reduce the size of the optical device to a millimeter scale, and integrate a wireless charging module therewith. Although these effectively reduce the plague to the animals and improve the reliability of the experimental results, an invasive implantation process is still needed.

In the treatment of diabetes, the patients are required to be injected with insulin regularly and the blood glucose level is stabilized by one or more hypoglycemic agents in combination. In the pathophysiological process of type 2 diabetes, the patients are not only relatively deficient of insulin, but also resistant to insulin. Therefore, the traditional insulin injection for the treatment of type I diabetes has limited effect on type 2 diabetes. In view of the complicated pathogenic factors of type 2 diabetes, the mechanism of insulin resistance has not yet been elucidated. Therefore, there is an urgent need to find a new method to avoid insulin resistance and thereby accurately control the blood glucose level.

CN108686208A discloses a non-invasive near-infrared light-controlled nanomaterial for repairing damaged nerves, where the non-invasive near-infrared light-controlled nanomaterial disclosed is an upconversion fluorescent nanomaterial; and discloses use of the upconversion fluorescent nanomaterial in neural repair. However, due to the optical properties and surface chemical modification of nanomaterials are different from CN108686208A and the complexity of the internal environment of organisms, whether the upconversion fluorescent nanomaterial is effective in the treatment of diabetes is still unknown.

### SUMMARY OF THE INVENTION

To solve the above technical problems, an object of the present invention is to provide a non-invasive near-infrared light-controlled nanomaterial for the treatment of diabetes. The present invention discloses the new use of upconversion fluorescent nanomaterial in non-invasive diabetes treatment. In the treatment of diabetes, there is no need to surgically implant invasive optical fibers in animals, and the upconversion nanomaterial in an organism is excited by near-infrared light with high tissue penetrability. The upconversion material converts the light of near-infrared band into visible light, to activate light-sensitive proteins. This enables the remote control of intracellular glucose metabolism-related signaling pathways independent of insulin with high temporal-spatial resolution, to promote the glycogen synthesis, inhibit the gluconeogenesis, and lower the blood glucose level.

The present invention provides use of the upconversion fluorescent nanomaterial in the preparation of a tool for the treatment of diabetes, where the upconversion fluorescent nanomaterial comprises an inorganic nanomaterial doped with rare earth elements, and a layer of water-soluble polymer and molecules targeting liver cells, which is attached onto the surface of the nanomaterial.

Preferably, there are many types of tools for the treatment of diabetes, for example, one that can be taken up or adsorbed by the designated cells of the damaged site by molecular targeting after the phase inversion by surface modification; or one that can be complexed with a biological material and located directly on the damaged site by surgery, where the upconversion material is excited to complete the repair by applying light outside the body.

The upconversion fluorescent nanomaterial functions to, during the repair process, provide light of required wavelength in vivo by using near-infrared light (NIR) after targeting to a specific site or cell. The upconversion fluorescent nanomaterial of the present invention converts light in the near-infrared band into visible light.

Preferably, the method of using the above tool includes the following steps:

(1) transfecting an organism with plasmids carrying a light-sensitive protein, to allow the plasmids carrying the light-sensitive protein to express in liver cells of the organism; and

(2) injecting the upconversion fluorescent nanomaterial into the organism treated in Step (1), and irradiating the liver of the organism with near-infrared light.

Preferably, in Step (1), the photosensitive protein is any of CIBN and CRY2, LOV, UVR8, or PhyB and PIF. Preferably, the plasmids carrying the light-sensitive protein is mCherry-CRY2-iSH and CIBN-CAAX.

The blue light-responsive protein cryptochrome 2 (CRY2) and the transcription factor CIBN are a protein pair commonly used in optogenetic research. In the present invention, UCNP with unique light conversion ability (converting near-infrared light into blue light) and a fusion protein molecule (CRY2/CIBN) are combined, to selectively activate the PI3K/AKT signaling pathway remotely. CIBN is fused with a small segment of cell membrane locating sequence CAAX, and its pairing molecule CRY2 is fused with the red fluorescent protein mCherry and the iSH2 domain for binding the endogenous PI3K catalytic subunit p110α. By means of the light conversion effect of UCNP, near-infrared light can penetrate deeply into the tissue and is upconverted to activate the mCherry-CRY2-iSH2 fusion protein. During the process of CRY2 binding to CIBN immobilized on the cell membrane, the catalytic subunit p110α of PI3K is also brought to the vicinity of the cell membrane to promote the PIP2 phosphorylation into PIPS, and then activates the PI3K/AKT pathway to control glucose metabolism by promoting the glycogen synthesis and inhibiting the gluconeogenesis. Therefore, the method of the present invention ameliorates the blood glucose level in the insulin-resistant type 2 diabetes model by triggering the cell signaling pathway by near-infrared light.

Preferably, in Step (2), the wavelength of the near-infrared light is between 0.7 μm and 2.5 μm and preferably 800-1000 nm, and the power is 0.5-2 W/cm². The liver is irradiated once a day, for 1-5 minutes each time. More preferably, the wavelength of the near-infrared light is 980 nm. Near-infrared light is used as the excitation light, which has a significantly improved tissue penetration depth.

Preferably, in Step (2), a single dose of the upconversion fluorescent nanomaterial is 5 mg/kg body weight, once a day.

Preferably, the upconversion fluorescent nanomaterial is used to lower the blood glucose level.

Preferably, the diabetes is type 2 diabetes.

Preferably, the molecule targeting liver cells is selected from glycyrrhetinic acid (GA) and/or glycyrrhizic acid. The weight ratio of the rare earth element-doped inorganic nanomaterial to the molecule targeting liver cells is 1:0.02-0.1. GA is a small molecule compound that can selectively recognize and target a receptor on the surface of liver cells.

Preferably, the water-soluble polymer is selected from polyethylene glycol (PEG), polyacrylic acid, polyethyleneimine and any combination thereof; and preferably PEG.

Preferably, the weight ratio of the rare earth element-doped inorganic nanomaterial to the water-soluble polymer is 1:1-2.

In the present invention, the surface of the rare-earth element-doped inorganic nanomaterial is modified with PEG and GA to increase the in-vivo circulation time and the liver targeting ability of the rare-earth element-doped inorganic nanomaterial.

Preferably, the rare earth element-doped inorganic nanomaterial has a core-shell structure, where the core includes a first matrix material and rare earth ions, and the shell includes a second matrix material, in which the first matrix material and the second matrix material are independently selected from NaYF₄, NaGdF₄ and KYF₄, preferably NaYF₄; and the rare earth ions are Tm³⁺, Yb³⁺, Nd³⁺, Tm³⁺, Er³⁺, Ho³⁺, Eu³⁺, or Tb³⁺, preferably Tm³⁺. The upconversion fluorescent nanomaterial comprises lanthanide rare earth elements, which has the characteristic of converting near-infrared light into ultraviolet-visible light. Therefore, it can solve the existing problem that optogenetic proteins mainly respond to short-wavelength photons (ultraviolet light, blue light, and green light).

Preferably, the molar ratio of the first matrix material, the rare earth ions and the second matrix material is 1:0.4-0.6:1.

Preferably, the upconversion fluorescent nanomaterial is UCNP-PEG-GA, which is prepared by a method including the following steps:

synthesizing NaYF₄:Yb/Tm@NaYF₄ upconversion nanoparticles (UCNP) having a core-shell structure, then modifying the surface of the UCNP particles with polyacrylic acid (PAA) by carboxyl substitution, to convert the UCNP into the water phase, and then modifying with polyethylene glycol (PEG) and PEG-GA with liver cell targeting ability by EDC/NHS coupling, to obtain a UCNP-PEG-GA solution. PEG-GA represents glycyrrhetinic acid attached with polyethylene glycol.

By means of the above technical solution, the present invention has the following advantages.

1. The present invention discloses use of a non-invasive upconversion fluorescent nanomaterial in the preparation of a tool for the treatment of diabetes. Based on this technology, daily injection of insulin to stabilize the blood glucose and administration of one or more hypoglycemic agents with certain side effects can be avoided during the use. Compared with traditional optogenetics, there is no need to surgically implanting invasive optical fibers, thus avoiding a series of side effects caused by trauma and avoiding of the use of optical fibers, to improve the flexibility.

2. In the present invention, a non-invasive upconversion fluorescent nanomaterial with high tissue penetrability is used, which greatly improves the tissue penetration compared to visible light. The non-invasive near-infrared light-controlled nanomaterial is selectively enriched in the liver, and the glucose metabolism is remotely regulated by near-infrared light. The upconversion fluorescent nanoparticles and optogenetic technology are used to selectively activate the PI3K/AKT signaling pathway to form a non-insulin-dependent treatment of type 2 diabetes. This method has the advantages of rapid response (second level), deep tissue penetration (centimeter level), and adjustable light dose. It successfully achieves the regulation of glucose metabolism in vitro and in animals, and provides a possible alternative strategy developed for dealing with the challenge in the clinical treatment of type 2 diabetes.

3. By means of light control, the light dose and the site irradiated can be adjusted, and the degree of treatment by light irradiation can be flexibly adjusted according to the blood glucose level. At the same time, the damaged site can be precisely treated by the use of laser to avoid the impact on other tissues and organs.

The above description is only a summary of the technical solutions of the present invention. To make the technical means of the present invention clearer and implementable in accordance with the disclosure of the specification, the preferred embodiments of the present invention will be described in detail with reference to the accompanying drawings.

### BRIEF DESCRIPTION OF THE DRAWINGS

FIG. 1 schematically shows a synthesis route of UCNP-PEG-GA and transmission electron microscopy (TEM) images of various materials;

FIG. 2 shows the test results of dynamic light scattering of various materials;

FIG. 3 shows the fluorescence spectra, ultraviolet absorption spectra and potential test results of various materials;

FIG. 4 shows the test results of cell viability of various cells and the test results of Calcein-AM/PI double staining of viable and dead cells;

FIG. 5 shows the statistical results of uptake of different materials by various cells;

FIG. 6 schematically shows the membrane translocation of mCherry-CRY2-iSH2 fusion protein mediated with or without UMO+NIR and binding of CIBN CAAX;

FIG. 7 shows images from the red fluorescence channel before and after HepG2 cells are irradiated with NIR;

FIG. 8 shows the pixel intensity distributions of mCherry fluorescence along the white arrows in c1-c2 of FIG. 7;

FIG. 9 shows images from the red fluorescence channel before and after HepG2 cells transfected with mCherry-CRY2-iSH2 plasmid alone are irradiated with NIR;

FIG. 10 shows the results of cell immunofluorescence staining in various test groups and the semi-quantitative statistical results of fluorescence images;

FIG. 11 shows multicolor fluorescence images of HepG 2 cells pretreated with GlcN at various irradiation times of NIR;

FIG. 12 shows the semi-quantitative statistical results of FIG. 11;

FIG. 13 shows the Western blot test results of AKT, p AKT, GSK 3β, p GSK 3β, FOXO 1 p FOXO 1 and GAPDH in various test groups;

FIG. 14 shows the test results of glucose level, cell survival rate, glycogen content, glucose production, and glycogen synthesis and inhibition of gluconeogenesis in the presence of a PI3K inhibitor in various test groups;

FIG. 15 compares the fluorescence intensities in the liver of mice injected with different nanomaterials through the tail vein and shows the distribution of the same nanomaterial in different organs after injection;

FIG. 16 shows the confocal fluorescence images in time course experiment of a frozen section of mouse liver;

FIG. 17 schematically shows the in-vivo experimental procedure of UMO, the test results of blood glucose level and glucose tolerance in various test groups of mice and the test results of liver glycogen content;

FIG. 18 shows the results of periodic acid-Schiff staining of mouse liver in various test groups; and

FIG. 19 shows the test results of phosphorylation levels of AKT and GSK3β in the liver of mice with type 2 diabetes after treatment with UMO+NIR.

### DETAILED DESCRIPTION OF THE PREFERRED EMBODIMENTS

The specific embodiments of the present invention will be described in further detail with reference to embodiments. The following embodiments are intended to illustrate the present invention, instead of limiting the scope of the present invention.

In the following examples and drawings of the present invention, unless otherwise specified, UMO refers to the UCNP-PEG-GA material prepared in the present invention conjugated with a fusion protein molecule (CRY2/CIBN), which does not receive NIR laser irradiation.

UMO+NIR refers to the implementation of NIR laser irradiation on UMO.

Unless otherwise specified, the NIR irradiation in the following examples occurs at a wavelength of 980 nm and a power of 1.2 W/cm² for 3 min each irradiation.

### Example 1: Synthesis of UCNP-PEG-GA

### 1. Synthesis of NaYF₄:Yb/Tm@NaYF₄ Upconversion Nanoparticles (UCNP) with Core-Shell Structure

The NaYF₄:Yb/Tm@NaYF₄ upconversion nanoparticles were synthesized by solvothermal method.

First, the core NaYF₄:Yb/Tm was synthesized. YCl₃ (0.695 mmol, 135.78 mg), YbCl₃ (0.30 mmol, 83.82 mg) and TmCl₃ (0.005 mmol, 1.38 mg) were weighed respectively on a balance, and added to a 50 mL three-necked flask. Then oleic acid (12 mL) and octadecene (15 mL) were added. After the three-necked flask was fixed on a hot plate, nitrogen was blown into the reaction device for 5 min to remove the air, and then the reaction system was heated. The reaction system was maintained at 160° C. with magnetic stirring for 0.5 h to dissolve the reactants and remove excess oxygen and water in the reaction system. The heating was stopped. After the reaction system was cooled to room temperature, a prepared methanol solution (10 ml) containing ammonium fluoride (4 mmol, 148 mg) and sodium hydroxide (2.5 mmol, 100 mg) was added dropwise to the reaction system via a syringe, and the reaction system was magnetically stirred at room temperature for 2 h, heated and maintained at 100° C. for 15 min to remove excess methanol in the reactants, and then further heated to 300° C. and reacted at this temperature for 1 h. After the reaction was completed, the heating was stopped. The reaction system was cooled to room temperature, and the product obtained after the reaction was washed three times by mixing with ethanol in a volume ratio of 1:3, and then separating by centrifugation at 10,000 rpm. The resulting precipitate was re-dispersed in cyclohexane (18 mL), to obtain NaYF₄:Yb/Tm upconversion nanoparticles (hereinafter referred to as UCNP core).

Next, NaYF₄:Yb/Tm@NaYF₄ upconversion nanoparticles with a core-shell structure were synthesized. Analogues to the above process, YCl₃ (0.695 mmol, 135.78 mg) was weighed and added to a 50 ml three-necked flask, and oleic acid (12 mL) and octadecene (15 mL) were added. Nitrogen was blown into the reaction device to remove the air, and then the reaction device was maintained at 160° C. with stirring for 30 min to dissolve the reactants and remove water and oxygen. The heating was stopped. After the reaction solution was cooled to 80° C., the solution (6 ml) of NaYF₄:Yb/Tm obtained above in cyclohexane was added to the reaction system through a syringe, and then heated to 120° C. to evaporate the cyclohexane in the mixed solution. The heating was stopped. After the reaction solution was cooled to room temperature, a methanol solution (10 mL) containing ammonium fluoride (4 mmol, 148 mg) and sodium hydroxide (2.5 mmol, 100 mg) was added dropwise. After stirring for 2 h at room temperature, the temperature was raised to remove methanol, and then raised to 75° C. for 10 min to remove the solvent methanol. After methanol was removed, the reaction solution was heated to 300° C. and reacted at this temperature for 1 h. After cooling, the product was washed three times with ethanol and then dissolved in cyclohexane to obtain NaYF₄:Yb/Tm@NaYF₄ upconversion nanoparticles with a core-shell structure (hereinafter referred to as UCNP core-shell).

### 2. Modification of Upconversion Nanoparticles UCNP

The surface of UCNP synthesized in Step 1 was modified with polyacrylic acid (PAA) by carboxyl substitution to convert the UCNP into the water phase. PAA replaced the oleic acid on the surface of UCNP through the ligand exchange method, thereby coating the surface of UCNP. Specifically, excess solution of PAA with a molecular weight of 2000 was dripped into a solution of UCNP in cyclohexane while ultrasonicated for 1 h. During this process, the solution was persistently blown with a pipette to uniformly mix the solution. Then the reactor was stirred for 8 h in a water bath at 50° C. The solution was allowed to stand, and the lower aqueous phase was separated by a separatory funnel. After washing three times with ethanol and water by centrifugation at 14000×g, the precipitate was re-dissolved in ultrapure water to obtain water-soluble UCNP-PAA.

By EDC/NHS coupling, GA was firstly linked to PEG (Boc-NH-PEG-NH₂) with a molecular weight of about 2400 having two terminal amino groups, one of which was protected by Boc. GA (47 mg) was dissolved in dichloromethane (5 mL), and then added dropwise to a dichloromethane solution (5 mL) containing DCC and NHS (at a molar ratio of GA:DCC:NHS=1:2:1.2). After stirring for 30 min, Boc-NH-PEG-NH₂ (240 mg) was added and stirred for 24 h. The carboxyl group in the GA molecule reacted with the amino group in the Boc-NH-PEG-NH₂ molecule by DCC/NHS coupling, to obtain Boc-NH-PEG-GA. After the reaction was completed, trifluoroacetic acid (2 mL) was added to remove the Boc at the amino end of the PEG. Pure NH₂-PEG-GA was obtained after the product was ultrafiltered and lyophilized.

The UCNP-PAA prepared above was reacted with NH₂-PEG-GA by EDC/NHS coupling by linking the amino group in NH₂-PEG-GA to the carboxyl group in the PAA molecule by NHS/EDC coupling. To balance the liver cell targeting ability and the water solubility of the nanomaterial, water-soluble PEG was also attached to the surface of UCNP. Specifically, NH₂-PEG-GA and NH₂-PEG-NH₂ were modified onto the surface of UCNP in a molar ratio of 1:3. EDC:NHS=1:0.6 were dissolved in ultrapure water, and added dropwise to the UCNP-PAA aqueous solution. After stirring for 30 min, a mixed aqueous solution of NH₂-PEG-GA and NH₂-PEG-NH₂ was added, and continuously stirred for 24 h. After the reaction was completed, the reaction product was washed three times with ultrapure water by centrifugation, and re-dispersed in ultrapure water or PBS to obtain water-soluble UCNP-PEG-GA.

In addition, UCNP-PEG was prepared as a control following the above method, in which after UCNP-PAA was synthesized, it was only reacted with NH₂-PEG-NH₂ by EDC/NHS coupling, and not with NH₂-PEG-GA. As a result, water-soluble UCNP with only PEG attached to the surface was obtained.

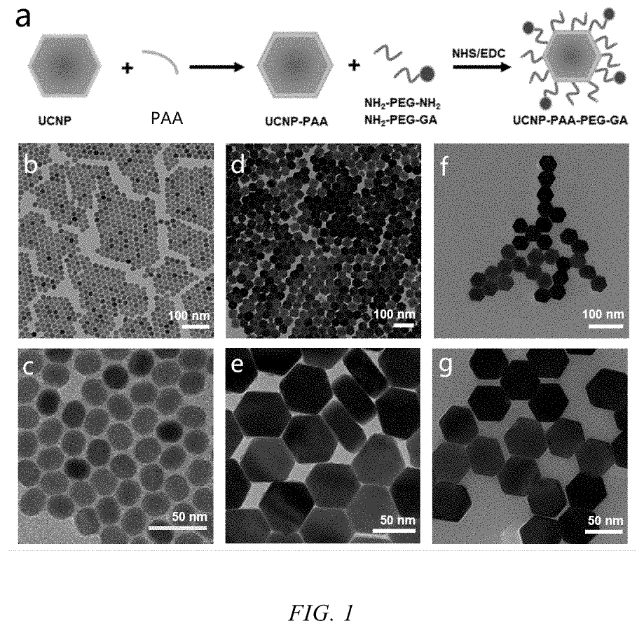

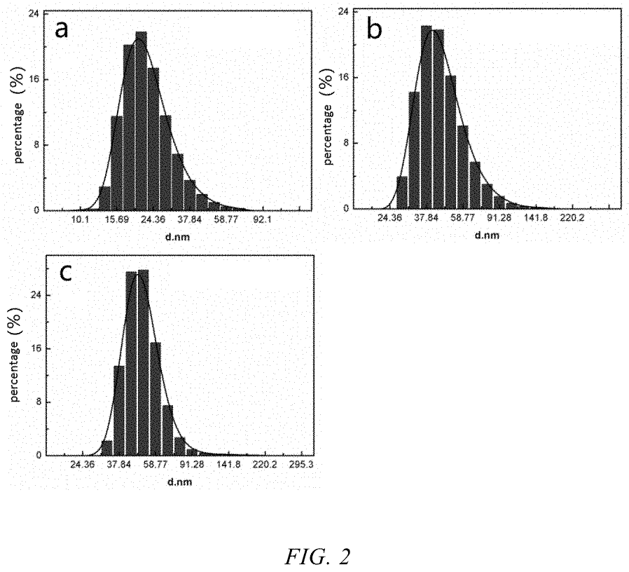

FIG. 1a schematically shows a synthesis route of UCNP-PEG-GA. FIGS. 1b-g show TEM images of UCNP core, UCNP core-shell and UCNP-PEG-GA. It can be seen from the figures that the average particle size of UCNP core is about 22 nm (FIGS. 1b-c), and the particle sizes of UCNP core-shell (FIGS. 1d-e) and UCNP-PEG-GA (FIGS. 1f-g) are about 45 nm. Their particle sizes were further confirmed by dynamic light scattering (DLS) (FIG. 2). FIGS. 2a, b and c show DLS images of UCNP core, UCNP core-shell and UCNP-PEG-GA, respectively.

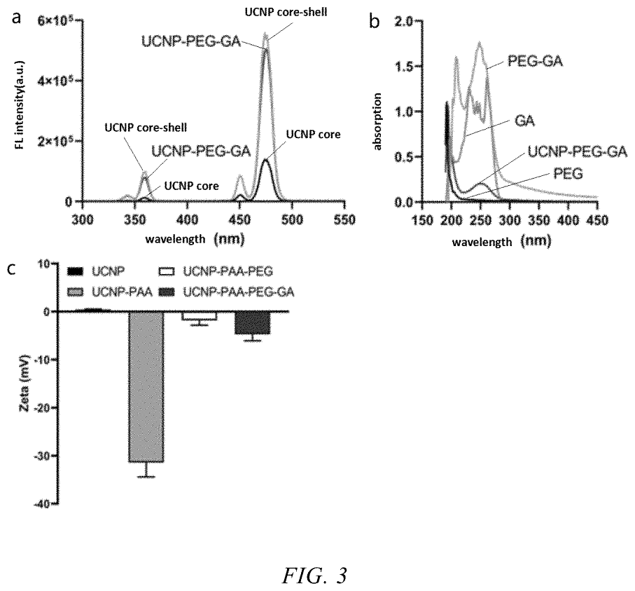

From the fluorescence spectrum (FIG. 3a), it can be seen that compared with the UCNP core, the emission intensity of the UCNP core-shell at 475 nm is increased by about 4 times. In addition, surface modification of UCNP does not have a noticeable impact on the upconversion efficiency of UCNP. In the ultraviolet absorption spectrum (FIG. 3b), both PEG-GA and UCNP-PEG-GA have an obvious absorption peak attributed to GA at 250 nm, while the simple PEG solution has no obvious absorption in the corresponding wavelength range, proving that PEG-GA was successfully synthesized and then successfully modified onto the surface of the UCNP core-shell. In addition, the potential of the nanomaterial also changes significantly during the modification process. For example, UCNP modified with PAA has a potential of −31.4 mV, UCNP-PAA modified with NH₂-PEG-NH₂ has a potential of −1.9 mV, UCNP-PAA modified with NH₂-PEG-GA has a potential of −4.7 mV (FIG. 3c). These results effectively prove that the surface of the UCNP particles is successfully modified with PEG or PEG-GA through the above-mentioned coupling strategy.

### Example 2: Cell Model and Related Research

HepG2 cells were purchased from ATCC, and HUVEC cells were provided by Tang Zhongying Hematology Research Center. The cells were cultured in a DMEM medium containing 10% FBS and 25 mM glucose at 37° C. in a humidified environment with 5% CO₂.

In order to obtain a HepG2 cell model of insulin resistance, HepG2 cells were induced and cultured in a DMEM (low-glucose) medium containing 18 mM glucosamine (GlcN) and 5 mM glucose for 18 h to obtain a HepG2 cell model of insulin resistance. The plasmid transfection was done by using the jetPRIME (Polyplus) reagent. When the cells were grown to 50% confluence, the plasmids CIBN-CAAX and mCherry-CRY2-iSH were added to a transfection buffer at a ratio of 1:1.2, and shaken for 5 s. Then, the transfection reagent (where 1 μg plasmid corresponds to 50 μL transfection buffer and 1 μL transfection reagent) was added, allowed to stand for 10 min, then added to a cell culture medium, and shaken uniformly. After 6 h, the medium was replaced by a fresh medium containing 200 μg/mL UCNP-PEG-GA. After 12 h of culture, the medium was replaced to remove excess UCNP-PEG-GA, and after 24 h of transfection, the transfection efficiency was observed and the cells were tested.

Before the immunofluorescence and western blotting assays, the cells were irradiated with near-infrared laser (980 nm, 1.2 W/cm², 3 min each time, 3-min interval, 3 times in total). The cells were then immobilized with 4% paraformaldehyde for 20 min at room temperature for immunofluorescence staining or collected by centrifugation for western blotting assay. In a control experiment involving the addition of a PI3K inhibitor, the cell culture medium was pretreated with a 10 μM small molecule inhibitor (LY294002) for 24 h, followed by subsequent related experiments.

To check the cell viability, the cells were inoculated in a 96-well plate at a density of 8000 cells per well. After the cells were adhered, the culture medium was replaced by culture media containing different concentrations of nanomaterial, and the cells were cultured for another 24 h. Then, the cell viability was detected by chemiluminescence using CellTiter-Glo® (CTG, Promega). 20 μL of the prepared CTG solution was added to each well of a 96-well plate. Then the plate was shaken on a shaker for 5 min, and allowed to stand for 5 min. The chemiluminescence of each well was read on a microplate reader, and converted into the cell survival rate of each well. The double staining test of viable and dead cells was done using the Calcein-AM/PI (YEASEN, #40747ES76) kit. The UCNP-PEG-GA nanomaterial was co-incubated with the cells for 24 h, and then the medium was removed. The cells were washed with PBS, and incubated at 37° C. for 15 min in a confocal dish added with 0.5 mL of Calcein-AM/PI staining reagent prepared according to the instructions. The cells were washed with PBS, and imaged under a confocal microscope (calcein channel (green): Ex: 490 nm; Em: 515 nm. PI channel (red): Ex: 535 nm; Em: 617 nm). Moreover, cells cultured with normal culture medium without adding any other materials were used as a control.

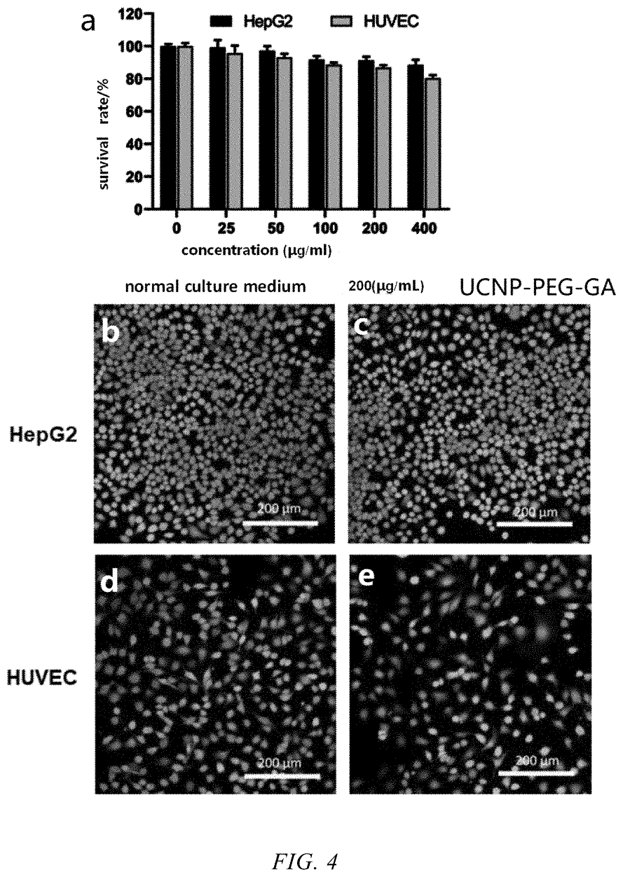

As shown in FIG. 4, it can be seen from the cell viability test results that when the concentration of UCNP-PEG-GA is as high as 200 μg/mL, no obvious cytotoxicity to HepG2 cells (liver cancer cells) and HUVEC cells (human umbilical vein endothelial cells) wad found (FIG. 4a). The Calcein-AM/PI double staining test of viable and dead cells further confirms the good biocompatibility of UCNP-PEG-GA (FIGS. 4b-e). As can be seen from FIGS. 4b-e, the medium added with 200 μg/mL UCNP-PEG-GA nanomaterial has the same viable cell count as the normal medium.

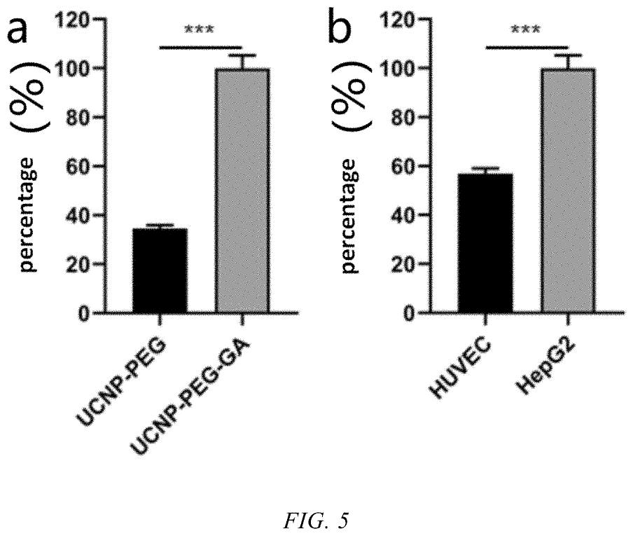

The uptake of UCNP-PEG-GA by the cells is obtained by measuring the level of rare earth element (Yb) contained in the cells by inductively coupled plasma mass spectrometry (ICP-MS). As shown in FIG. 5a, the uptake of UCNP-PEG-GA by HepG2 cells is about 3 times that of UCNP-PEG. In addition, due to the GA receptor on the surface of HepG2 cells, the UCNP-PEG-GA uptake by HepG2 cells is 2 times the uptake by HUVEC cells (FIG. 5b). In FIG. 5, *: P<0.05, **: P<0.01, ***: P<0.001. The above results prove that the surface modification of UCNP by PEG-GA promotes the uptake of nanoparticles by liver-derived cells.

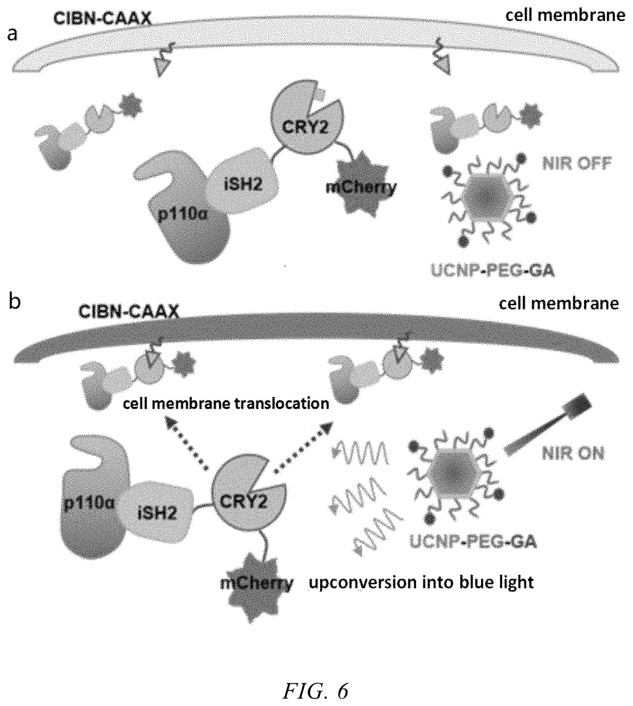

HepG2 cells were co-transfected with CIBN-CAAX and mCherry-CRY2-iSH2 plasmids at a weight ratio of 1:1.2. After 24 h, 200 μg/mL UCNP-PEG-GA was added and co-incubated with the cells. After 12 h, the medium was refreshed, followed by a stimulation experiment by near-infrared irradiation. As shown in FIG. 6, the mCherry-CRY2-iSH2 fusion protein is randomly distributed in the cytoplasm without stimulation (FIG. 6a). In the presence of UCNP-PEG-GA, the blue light produced by upconversion of NIR promotes the CRY2 molecule in the fusion protein to undergo a conformational change, to recognize and bind to the CIBN immobilized on the cell membrane, and quickly move to the vicinity of the cell membrane with the remaining fusion protein, which increases the red fluorescence signal on the cell membrane (FIG. 6b).

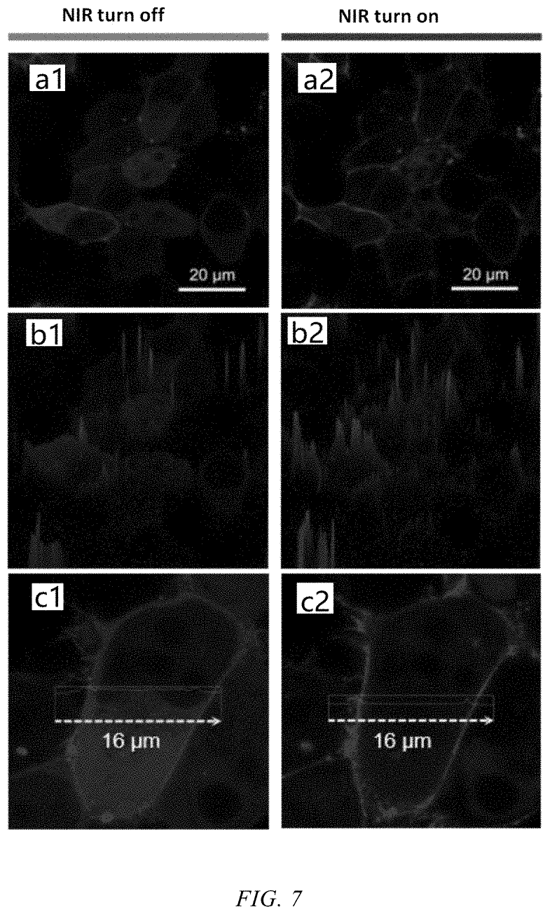

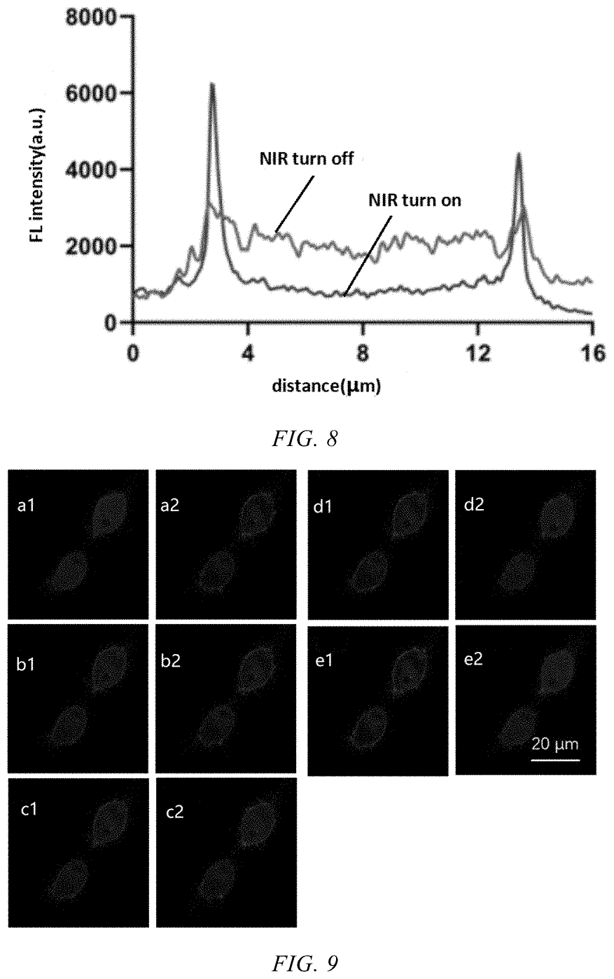

In FIG. 7, before and after HepG2 cells were irradiated with NIR (1 W/cm², 2 min), the cells were imaged from the red fluorescence channel mCherry. After NIR irradiation, the distribution of mCherry in the cells shows a significant shift from the cytoplasm to the cell membrane (FIGS. 7a1-a2). From the 2.5D perspective of FIG. 7a1, after NIR irradiation, the fluorescence peak intensity at the cell membrane increases significantly (FIGS. 7b1-b2). In FIGS. 7c1-c2, a HepG2 cell is selected as a representative example to study the details of membrane translocation of a protein. It can be seen that the fluorescence intensity in the cytoplasm is reduced due to the translocation of mCherry-CRY2-iSH2, while the fluorescence on the cell membrane is significantly enhanced by the binding of CRY2 to CIBN anchored on the cell membrane. In FIGS. 7C1-c2, the white dashed arrow running through HepG2 cells is provided to analyze the fluorescence distribution at the pixel points in the cell cytoplasm and on the cell membrane. The shape of the fluorescence curve like a plateau before irradiation shows that the mCherry-CRY2-iSH2 protein has a uniform distribution in the cells. After the cells are irradiated by NIR, the fluorescence distribution is in the form of a typical peak-valley shape, showing a significant nonuniformity in protein distribution between the cell membrane and the cytoplasm (FIG. 8).

In addition, in the control experiment, HepG2 cells are transfected with the mCherry-CRY2-iSH2 plasmid alone. Due to the lack of CIBN-CAAX expression on the cell membrane, even if HepG2 cells are exposed to NIR irradiation, the mCherry-CRY2-iSH2 fusion protein cannot be anchored to the cell membrane. The NIR laser (1 W/cm²) are turned on for 30 s, and then turned off for a certain period of time, as shown in FIG. 9 (scale bar: 20 μm). FIGS. 9a1, b1, c1, d1, and e1 respectively show the fluorescence images at 0 s, 2 s, 4 s, 8 s, and 30 s after the NIR laser is turned on. FIGS. 9a2, b2, c2, d2, and e2 respectively show the fluorescence images at 120 s, 300 s, 600 s, 900 s, and 1200 s after the NIR laser is turned off. Before and after NIR irradiation, there is no protein translocation between the cytoplasm and cell membrane of HepG2, which is clearly different from that observed in FIG. 7. In addition, the information shown in the image also proves that in the method of the present invention, the homo-oligomerization between CRY2 and CRY2 is negligible, because the cytoplasm of HepG2 cells does not contain any obvious fluorophore caused by NIR irradiation. It is pointed out in previous literatures that CRY2 itself will undergo homo-oligomerization under blue light irradiation, which seriously affects the CIBN/CRY2 interaction in optogenetic research. The homo-oligomerization between CRY2 may be inhibited in the method of the present invention due to the steric hindrance caused by the mCherry and iSH domains in the fusion protein. These results indicate that under NIR irradiation, the interaction between CIBN/CRY2 can be used to direct the directional translocation of specific proteins in HepG2 cells.

### Example 3: HepG2 Cell Model of Insulin Resistances and Related Research

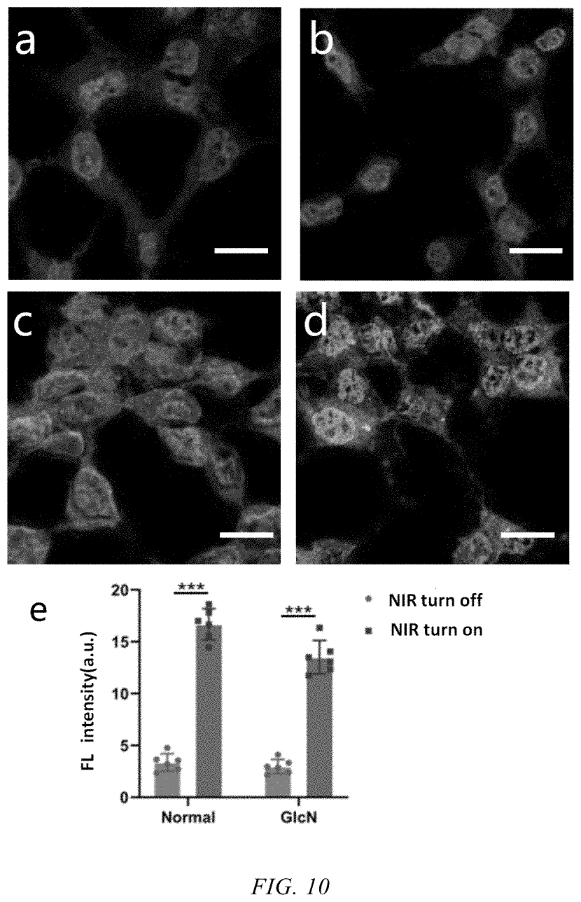

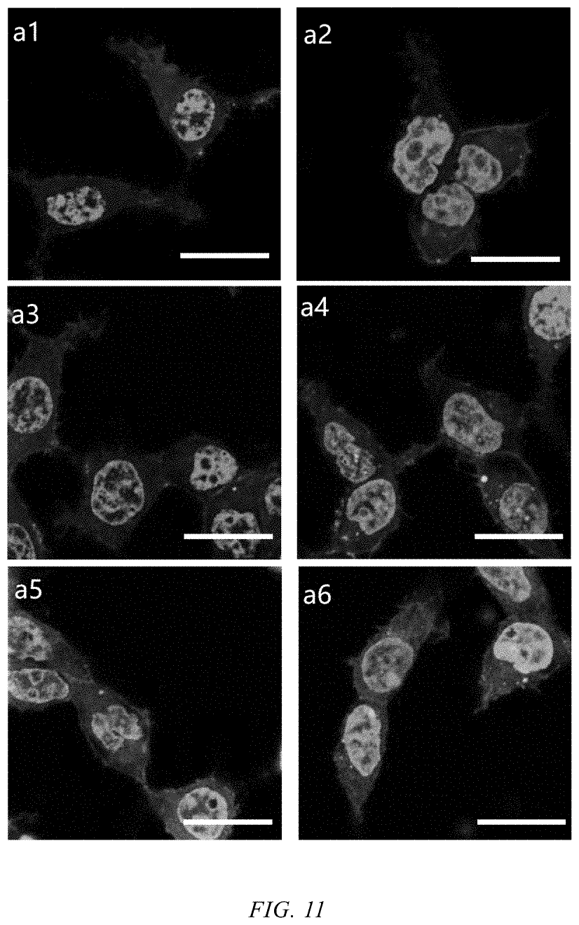

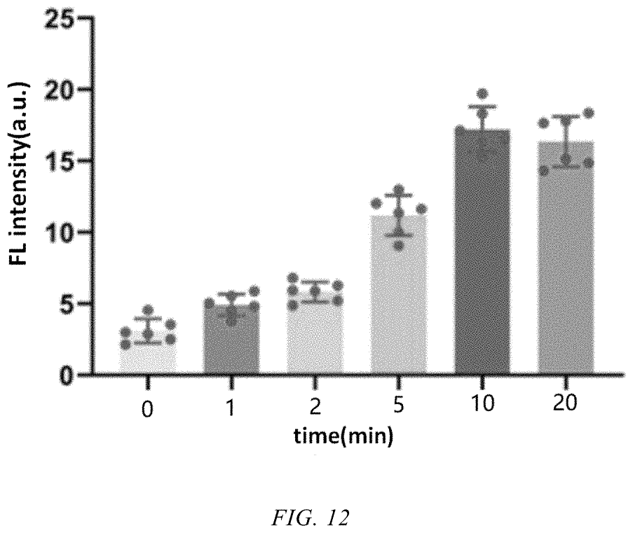

Glucosamine (GlcN) was used to induce an HepG2 cell model of insulin resistance to evaluate the effect of UMO in in-vitro experiment. FIGS. 10a-d show the test results of cell immunofluorescence staining. FIGS. 10a and b show the results of cell immunofluorescence staining of the normal control group and GlcN-induced group without NIR irradiation respectively. FIGS. 10c and d show the results of cell immunofluorescence staining of the normal control group and GlcN-induced group under NIR irradiation. In the figures, different colors are used to represent the fluorescence signals of mCherry, DAPI, and p-AKT. It can be seen from the cell immunofluorescence staining assay (FIG. 10) that the fluorescence signal intensity of p-AKT in both the normal group (FIG. 10c) and the GlcN-treated group (FIG. 10d) is enhanced after NIR irradiation compared with the control (the normal control group in FIG. 10a and GlcN control group in FIG. 10b). From the semi-quantitative statistical results of the fluorescence images, it can be seen that the p-AKT signal in normal HepG2 cells is enhanced by about 5 times compared with the control group. After treatment with GlcN, the insulin-resistant HepG2 cells also show about 4.5-fold enhancement compared to the control group (FIG. 10e). In a cell assay with a gradient of near-infrared irradiation times (FIG. 11a1-a6), the phosphorylation level of AKT is positively correlated with the time of NIR illumination, until the fluorescence intensity reaches saturation at about 10 min. The membrane translocation of mCherry-CRY2-iSH2 is also consistent with the previously observed result. FIGS. 11a1-a6 show the cell immunofluorescence images when the near-infrared irradiation time is 0 min, 1 min, 2 min, 5 min, 10 min, and 20 min. Semi-quantitative analysis of the fluorescence images of HepG2 cells after GlcN treatment confirms that the phosphorylation level of AKT shows an increase with the prolonged NIR illumination time (FIG. 12). In FIG. 12, *: P<0.05, **: P<0.01, ***: P<0.001.

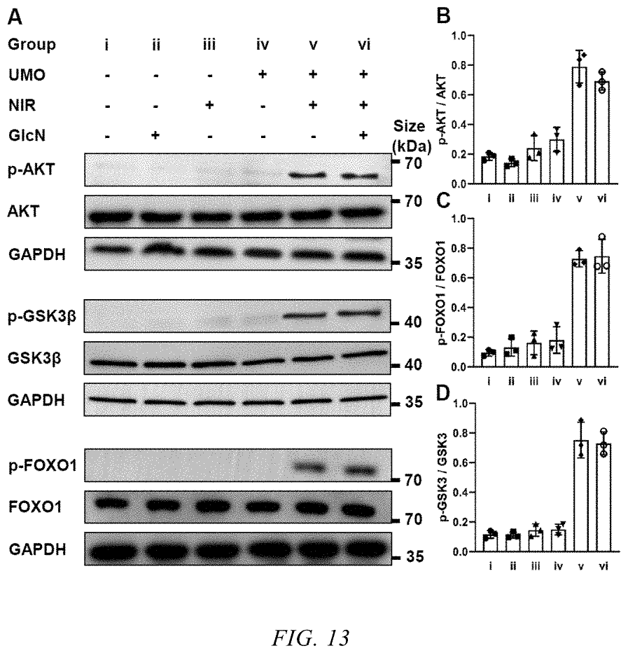

In the AKT signaling pathway, phosphorylated AKT can promote the phosphorylation of downstream protein molecules, including GSK3β and FOXO1, which coordinately regulate the blood glucose level by increasing the glycogen synthesis and inhibiting the gluconeogenesis. In Western blotting assay, different treatment conditions were applied to HepG2 cells, including GlcN (+/−), UMO (+/−) and NIR(+/−). As shown in FIG. 13A, UMO+/NIR+ can significantly increase the phosphorylation level of AKT (Ser 473), GSK3β (Ser 9) and FOXO1 (Ser 256) in HepG2 cells in both the normal group and the insulin resistant group. However, in the cell sample groups designated as ii, iii, and iv (UMO−/NIR−/GlcN+, UMO−/NIR+/GlcN−, UMO+/NIR−/GlcN−, respectively), the phosphorylation level of these proteins has no obvious change, compared with the control group (designated as i). This result provides a powerful support for the conclusion that UMO activation triggered by NIR leads to the phosphorylation of AKT, GSK3β and FOXO1. It can be seen from the statistical analysis results of Western blotting assay that after UMO activation, the phosphorylation levels of AKT, GSK3β and FOXO1 in insulin-resistant HepG2 cells are similar to the phosphorylation levels in normal HepG2 cells (FIGS. 13B-D)). This experiment clearly shows that the method of the present invention promotes the phosphorylation of key proteins in the PI3K/AKT signaling pathway in insulin-resistant HepG2 cells, thereby achieving the metabolic control of blood glucose in patients with type 2 diabetes.

The abnormal increase in blood glucose level in patients with type 2 diabetes is due to impaired regulation of glucose metabolism. The dysfunction of the insulin/PI3K/AKT/GSK3β pathway leads to the inability to synthesize glycogen with glucose. In addition, the abnormally active gluconeogenesis in patients with type 2 diabetes increases the production of glucose through the FOXO1/PEPCK/G6Pase pathway, thereby further deteriorating the metabolic balance of glucose. Therefore, in the treatment of type 2 diabetes, it is essential to ameliorate the blood glucose level by promoting the glycogen synthesis and inhibiting the excessive gluconeogenesis. By means of UCNP-PEG-GA treatment and near-infrared irradiation in combination, the change in glucose content in HepG2 cells that are treated with GlcN to simulate an insulin resistant environment is monitored. It can be seen in FIG. 14A that after insulin (100 nM) is added to the culture medium of normal HepG2 cells (insulin group), the glucose consumption in cells is increased by 2.5 times compared with the normal group (blank group). This result is expected, because insulin can promote glucose uptake and synthesis into glycogen in normal liver cells, but insulin is ineffective in the cells receiving GlcN treatment. However, the method of the present invention (UMO+NIR) can significantly promote glucose uptake in normal cells and insulin-resistant cells (85% relative to the effect on the normal cells under the activation of insulin). In the method, both normal and insulin resistant HepG2 cells can tolerate UMO+/−NIR treatment, and the cell viability has no significant loss compared to the blank group (FIG. 14B). Through further determination of the glycogen content in these cells, it is confirmed that glucose consumed in FIG. 14A is indeed converted to glycogen (FIG. 14C). In addition, the gluconeogenesis levels in cells exposed to different treatment conditions were also detected in a glucose-free medium. Compared with normal cells, the glucose production in GlcN pretreated cells is increased by 3.4 times (FIG. 14D), and insulin cannot inhibit the gluconeogenesis in GlcN pretreated cells. Apparently, UCNP-PEG-GA treatment combined with near-infrared irradiation can reduce the gluconeogenesis level in HepG2 cells in the insulin resistant group by half (FIG. 14D). Further, the present invention also confirms through experiments that the effect of UMO+NIR in promoting the glycogen synthesis and inhibiting the gluconeogenesis in insulin-resistant cells can be blocked by the PI3K inhibitor (LY294002) (FIGS. 14E and 14F), where the concentration of LY294002 is 10 μM. Therefore, these experimental results confirm that the method of the present invention can specifically promote the glycogen synthesis and reduce the gluconeogenesis through the PI3K/AKT pathway, thereby effectively controlling glucose metabolism.

### Example 4: In-Vivo Test and Test Results

Construction of mouse model of type 2 diabetes: The C57BL/6J mice used in the experiment were available from the Laboratory Animal Center of Soochow University. To induce a mouse model of type 2 diabetes, 6-week-old C57BL/6J mice were allowed to receive a low-dose injection of streptozotocin (STZ) and fed with a high-fat diet. This induction process simulated the pathological process of type 2 diabetes. Simply, 120 mg/kg body weight of STZ (dissolved in 10 mmol/L, citrate buffer pH 4.0) was injected into mice through the tail vein. STZ was used immediately after preparation, and stored on ice during the experimental process. After receiving STZ injection, the mice were fed with normal diet (14.7 kJ/g, 13 kcal %) for 3 weeks, and then with high-fat diet (21.8 kJ/g, 60 kcal % fat, Research Diets, #D12492) for 5 weeks. Mice with a blood glucose level of 20 mmol/L or higher were re-grouped randomly, which were successfully induced model mice of type 2 diabetes. The blood glucose level in mice was tested by the glucose test paper produced by Johnson & Johnson.

The mice were injected with UCNP-PEG or UCNP-PEG-GA (5 mg/kg body weight) dissolved in PBS through the tail vein. To study the distribution of UCNP-related material in mice, the heart, liver, spleen, lung, and kidney of the mice were dissected 48 h after injection, and subjected to upconversion imaging by modified Maestro™ EX (CRi. Inc., MA, USA) in-vivo imager. The in-vivo distribution of UCNP-related material was analyzed by the upconversion fluorescence signal intensity of UCNPs in various organs.

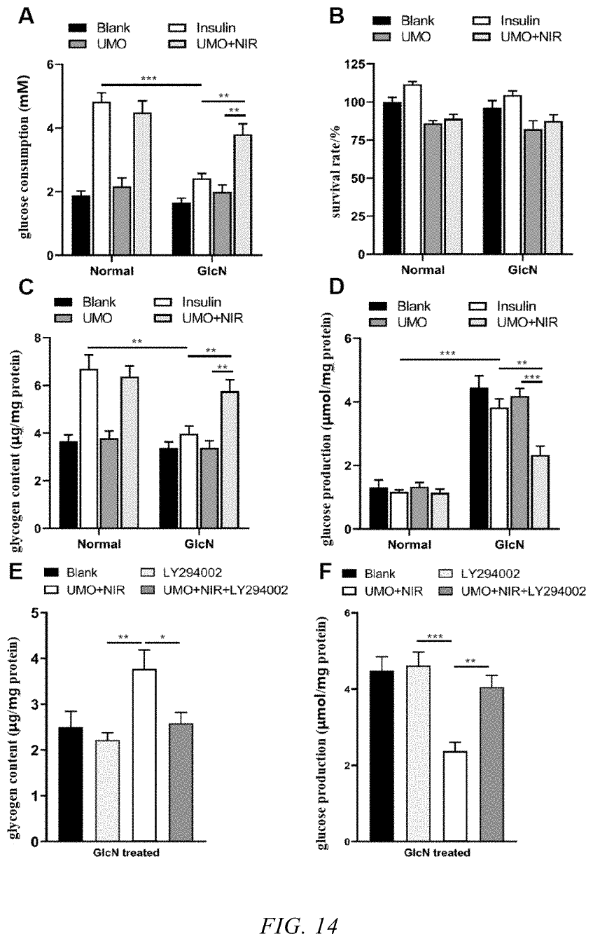

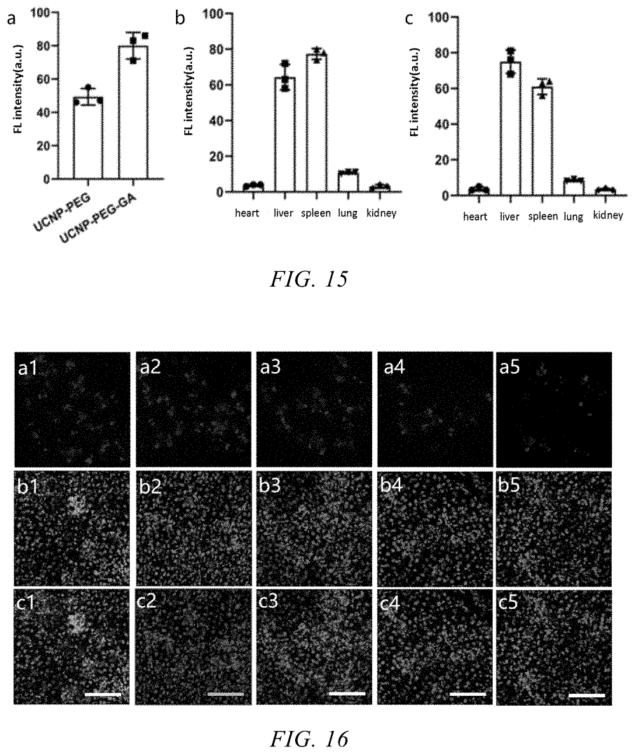

The semi-quantitative analysis results of fluorescence intensity of various organs of mice give the distribution trend of UCNP in these organs (FIGS. 15a-c). FIG. 15 compares the fluorescence intensities in the liver of mice injected with different nanomaterials through the tail vein, and FIG. 15b-c show the distribution of UCNP-PEG and UCNP-PEG-GA in various organs respectively. The distribution of UCNP-PEG in the spleen is slightly higher than that in the liver (FIG. 15b). For UCNP-PEG-GA (FIG. 15c), the distribution ratio between the liver and spleen is opposite to that of UCNP-PEG (FIG. 15b). These results prove that by means of GA molecules on the surface of nanoparticles, the targeting ability of UCNP-PEG-GA to the liver is approximately 2 times higher than that of UCNP-PEG (FIG. 15a).

### Example 5: In-Vivo Test and Test Results

The plasmids CIBN-CAAX and mCherry-CRY2-iSH were mixed and dissolved in PBS at a weight ratio of 1:1.2, to give a concentration of the mixed plasmids in PBS of 35 μg/mL. Each mouse established in Example 4 was injected with 2 mL of the plasmid solution through the tail vein. The entire injection process was completed within 8 sec. After the injection, the liver part of the mice was pressed to promote the expression of the plasmids in the mouse liver cells. To study the expression of the plasmids in various organs of mice, frozen sections of various organs of mice were prepared at different times. The cell nuclei were stained with DAPI, washed with PBS and mounted. The expression of mCherry in the optogenetic protein was observed under a confocal microscope to determine the expression level of the plasmids.

The results show that the liver shows the highest transfection efficiency compared with other organs, and the expression of CIBN/CRY2 in mouse liver reaches the peak one day after transfection (FIG. 16). Although the fluorescence signal of mCherry weakens with the extension of expression time, it can be seen from the image that these optogenetic proteins still maintain a considerably high expression for up to two weeks (FIG. 16). In FIG. 16, a1-a5 represent the expression of mCherry on day 1, day 2, day 4, day 8 and day 14 respectively, and b1-b5 represent the expression of DAPI on day 1, day 2, day 4, day 8 and day 14 respectively, and c1-c5 represent the expression of mCherry and DAPI on day 1, day 2, day 4, day 8 and day 14 respectively.

After the successful implantation of optogenetic elements was confirmed in mice, the system was used for the treatment of mice with type 2 diabetes. C57BL/6J mice were injected with a low-dose streptozotocin (STZ) and fed with a high-fat diet (HFD) to induce a type 2 diabetes model, and the changes in blood glucose level were monitored during the induction process. After 35 mice were induced by STZ/HFD for 5 weeks, the blood glucose level in 31 mice is higher than 20 mmol/L, which reflects the high rate (88.6%) of success construction of mouse model of type 2 diabetes.

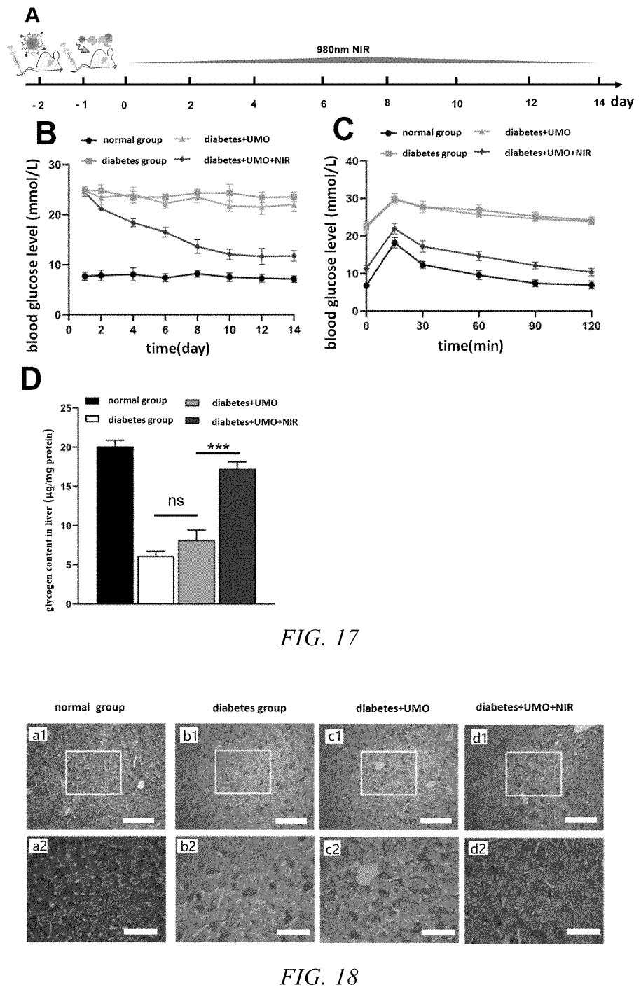

The in-vivo test procedure with UMO was shown in FIG. 17A. After mice of type 2 diabetes were injected with UCNP-PEG-GA and optogenetic plasmids respectively through the tail vein on day −2 and day −1, they received near-infrared irradiation treatment every day (980 nm laser, three minutes each time, 1.2 W/cm²) in a two-week treatment period. The blood glucose levels of all mice including those in the control group were monitored and recorded. As shown in FIG. 17B, during the treatment period, there are significant differences in the blood glucose levels of the mice in each group. The blood glucose level of mice with type 2 diabetes has been maintained at 24 mmol/L for two weeks, which is almost 3 times the blood glucose level of normal healthy mice. In contrast, when mice with type 2 diabetes are treated with UMO+NIR, the blood glucose level shows a clear trend of decline, and decreases from 24.4 mmol/L to 11.8 mmol/L (diabetes+UMO+NIR) in 14 days. As an important control, when mice with type 2 diabetes are implanted with UMO, but do not receive NIR irradiation (diabetes+UMO), the blood glucose level is still maintained at a level higher than 20 mmol/L. This indicates that in this method, UCNP-mediated near-infrared conversion is critical.

After NIR irradiation treatment, a glucose tolerance test was performed on mice in each group to further evaluate the therapeutic effect of the method of the present invention in the case of a sharp increase in blood glucose level. Before the glucose tolerance test, the mice were fasted for 12 h and received a near-infrared irradiation treatment. The mice were intraperitoneally injected with 2 g/kg body weight of a glucose solution, the blood glucose level of the mice was measured at 15, 30, 60, and 120 minutes after the injection. The tolerance to sharp increase of glucose level by the mice was analyzed by the blood vs glucose curve. As shown in FIG. 17C, the blood glucose level of mice in each group increases rapidly within 15 min after intraperitoneal injection of glucose. It can be observed that the “time-blood glucose” metabolic curve of mice in the diabetes+UMO+NIR group is similar to that of normal healthy mice, and the blood glucose level of the mice can be reduced to the baseline level within two hours in the two groups. The mice in the remaining two groups (diabetes, diabetes+UMO) cannot effectively alleviate the sharply elevated blood glucose (FIG. 17C). Quantitative analysis of the glycogen content in the liver of mice shows that the glycogen synthesis efficiency in the liver of mice with type 2 diabetes is only 30-40% of that of normal healthy mice. It is worth noting that compared with normal healthy mice, UMO+NIR treatment can restore the glycogen synthesis efficiency to 90% in the liver of mice with diabetes (FIG. 17D).

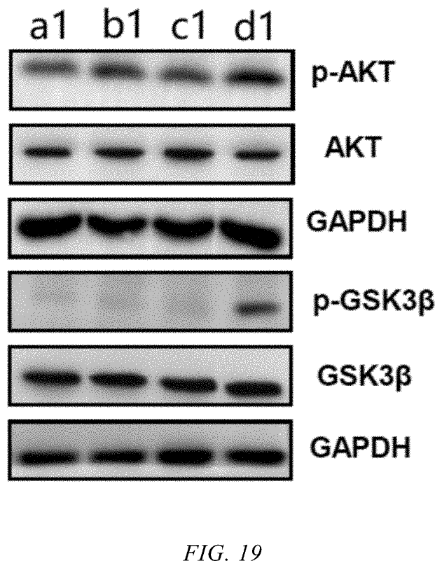

The results of periodic acid-Schiff (PAS) staining of mouse liver show (FIG. 18) that treatment of mice with type 2 diabetes with UMO+NIR can restore the glycogen storage level in the liver. As a negative control, the glycogen content in the liver of mice with diabetes that are untreated or only transplanted with UMO but did not receive NIR irradiation is significantly lower than that of normal healthy mice or mice returned to normal. In FIG. 18, a2-d2 are enlarged views corresponding to the a1-d1 boxes, respectively, where the scale bar in FIGS. 18a1-d1 is 100 μm, and the scale bar in a2-d2 is 50 Western blotting assay of mouse liver lysates was also used to study the in-vivo changes of key proteins in the process of glucose metabolism. The phosphorylation levels of AKT and GSK3β in the liver of mice with type 2 diabetes receiving UMO+NIR treatment are significantly increased compared to other controls (FIG. 19). These experiments finally show that the in-vivo UMO+NIR treatment can remotely restore the function of liver cells in the regulation of glucose metabolism by PI3K/AKT signaling pathway without the need for insulin injections.

In summary, the present invention develops a new method for remotely ameliorating the blood glucose level in type 2 diabetes model through near-infrared upconversion-mediated optogenetics. It mediates the activation of PI3K/AKT pathway in a non-insulin-dependent manner with the characteristics of rapid response, deep tissue penetration, and adjustable light dose. Based on this, the control of glucose metabolism level in in-vitro and in-vivo experiments is successfully achieved. This UMO-based method can be flexibly applicable to other important signaling pathways, such as NF-κB and MAPK signaling pathways, to solve immune and inflammation-related diseases. The UMO+NIR method of the present invention is essentially a non-invasive technique with deep tissue penetrability, which can realize remote control of intracellular signaling pathways with high temporal-spatial resolution. This new technology greatly enriches the optogenetic toolbox in signaling pathway research, and also provides new solutions for traditional clinical treatments.

While preferred embodiments of the present invention have been described above, the present invention is not limited thereto. It should be appreciated that some improvements and variations can be made by those skilled in the art without departing from the technical principles of the present invention, which are also contemplated to be within the scope of the present invention.

1. Use of an upconversion fluorescent nanomaterial in the preparation of a tool for the treatment of diabetes, wherein the upconversion fluorescent nanomaterial comprises an inorganic nanomaterial doped with rare earth elements, and a layer of water-soluble polymer and molecules targeting liver cells, which is on the surface of the nanomaterial.

2. The use according to claim 1, wherein the method of using the tool comprises steps of:
(1) transfecting an organism with plaimids carrying a light-sensitive protein, to allow the plasmids carrying the light-sensitive protein to express in liver cells of the organism; and
(2) injecting the upconversion fluorescent nanomaterial into the organism treated in Step (1), and irradiating the liver of the organism with near-infrared light.

3. The use according to claim 2, wherein in Step (1), the light-sensitive protein is any of CIBN and CRY2, LOV, UVR8, or PhyB and PIF.

4. The use according to claim 2, wherein in Step (2), the wavelength range of the near-infrared light is between 0.7 μm and 2.5 μm.

5. The use according to claim 1, wherein the upconversion fluorescent nanomaterial is used to lower the blood glucose level.

6. The use according to claim 1, wherein the diabetes is type 2 diabetes.

7. The use according to claim 1, wherein the molecule targeting liver cells is selected from glycyrrhetinic acid and/or glycyrrhizic acid; and the weight ratio of the rare earth element-doped inorganic nanomaterial to the molecule targeting liver cells is 1:0.02-0.1.

8. The use according to claim 1, wherein the water-soluble polymer is selected from the group of polyethylene glycol, polyacrylic acid, polyethyleneimine and any combination thereof; and the weight ratio of the rare earth element-doped inorganic nanomaterial to the water-soluble polymer is 1:1-2.

9. The use according to claim 1, wherein the rare-earth element-doped inorganic nanomaterial has a core-shell structure, where the core comprises a first matrix material and a rare earth ion, and the shell includes a second matrix material, the first matrix material and the second matrix material are independently selected from NaYF₄, NaGdF₄ or KYF₄, and the rare earth ion is Yb³⁺, Nd³⁺, Tm³⁺, Er³⁺, Ho³⁺, Eu³⁺ or Tb³⁺.

10. The use according to claim 9, wherein the molar ratio of the first matrix material, the rare earth ion and the second matrix material is 1:0.4-0.6:1.
